(19) United States
(12) Patent Application Publication
(10) Pub. No.: US 2011/0172432 A1
(43) Pub. Date: Jul. 14, 2011

(54) METHOD FOR THE SYNTHESIS OF HETEROGENEOUS PALLADIUM CATALYSTS, CATALYSTS OBTAINED AND USE OF SAME

(75) Inventors: Claude Le Drian, Mulhouse (FR); Jean-Michel Becht, Brunstatt (FR); Stéphane Schweizer, Habsheim (FR)

(73) Assignees: UNIVERSITE DE HAUTE ALSACE, MULHOUSE (FR); CENTRE NATIONAL DE LA RECHERCHE SCIENTIFIQUE, PARIS (FR)

(21) Appl. No.: 12/596,388

(22) PCT Filed: Apr. 11, 2008
(86) PCT No.: PCT/FR2008/050639

§ 371 Date: Apr. 27, 2010

(30) Foreign Application Priority Data
Apr. 16, 2007 (FR) 0754500

(51) Int. Cl. B01J 31/06; C07C 45/61; C07D 213/16; C07D 401/04; C07D 333/10; C07D 411/04; C07C 1/32

(52) U.S. Cl. 546/268.1; 502/159; 568/316; 546/352; 549/80; 549/59; 585/469

(57) ABSTRACT

The invention relates to the field of chemistry, especially organic chemistry, and more specifically the field of heterogeneous palladium catalysts used to catalyse chemical reactions involving the formation of carbon-carbon bonds. The invention also relates to a method for synthesising a heterogeneous palladium catalyst that can catalyse a C—C coupling reaction, the method essentially including steps of providing a solid substrate onto which groups of formula —PR₁R₂, wherein R₁ is an optionally substituted alkyl group, or an optionally substituted cycloalkyl group, et R₂ is an optionally substituted aryl group or an optionally substituted heteroaryl group, have been covalently bonded, and incorporating a catalytically effective amount of palladium into the resulting substituted substrate. The invention further relates to the resulting catalysts and to the uses thereof in C—C coupling reactions.

This invention relates to the field of chemistry, in particular organic chemistry, and more particularly the field of heterogeneous palladium catalysts that are used to catalyze chemical reactions that involve the formation of carbon-carbon bonds, in other words carbon-carbon coupling reactions.

Examples of such reactions are known under the names of Suzuki, Heck or Sonogashira reactions.

One of the objectives of organic synthesis is to carry out chemical reactions that minimize the purification stages that are necessary for obtaining a product in accordance with increasingly strict standards. Furthermore, an ever more intensive ecological desire to reduce waste reorients the methods for synthesis of numerous products. Finally, a requirement that is linked to the concept of long-lasting development consists in minimizing the quantities of reagents that are used.

In the field of reactions that are catalyzed by transition metals, among the traditional catalysts, it is necessary to distinguish the heterogeneous catalysts from the homogeneous (or soluble) catalysts. The first can be recovered easily, but the second offer much broader synthetic possibilities. Numerous works have been dedicated to the development of increasingly complex soluble catalysts. They are often expensive, however, and frequently lead to the presence of small quantities of metal derivatives in the reaction product, which requires additional purification stages that are often difficult to accomplish. In addition, the metal should, in general, be recovered, and wastes should be totally removed from it. The development of new heterogeneous catalysts that have the properties of homogeneous catalysts is the direct consequence of these economic criteria (reduction of costs owing to the reuse of an expensive catalyst and a simplified purification of the reaction products) and these ecological criteria (reduction of the quantity of metal that is present in the waste). The metal that is used is thus found almost entirely in the catalyst that is recovered at the end of the reaction.

In the particular case of palladium, it is necessary to emphasize that this metal has a high price (8,200ε per kg in October 2006). In addition, a shortage of this metal linked to the difference between a more or less constant production and applications whose number continues to grow in fields as varied as jewelry, automobiles and chemistry begins to be felt more and more. It is therefore reasonable to believe that its price will remain high or will rise even more in the years to come.

A current great challenge is therefore to heterogenize the soluble palladium catalysts by fixing them to a solid substrate (mineral or organic), whereby the purpose is ultimately to preserve both the ease of use of heterogeneous catalysts and the synthetic potentialities of recent homogeneous catalysts.

Palladium is the transition metal that has one of the strongest, and even the strongest, synthetic potential for the creation of new carbon-carbon bonds. The Suzuki reaction is a pallado-catalyzed coupling and constitutes a particularly effective tool for the creation of aryl-aryl or aryl-vinyl bonds. The thus produced molecules often constitute basic structures for the preparation of more complex molecules that find applications in numerous very varied fields such as pharmacochemistry, agrochemistry, semi-conductor materials, . . . .

The Suzuki reaction consists in reacting an aryl halide with a vinyl- or aryl-boronic acid. The aryl bromides, and especially the aryl iodides are coupling partners of choice for performing this transformation. During recent years, the use of aryl chlorides for Suzuki couplings, less expensive but also much less reactive than their brominated or iodized analogs, has given rise to great interest. Several homogeneous catalysts that make it possible to effectively couple an aryl chloride and an arylboronic acid have been described in the literature. These homogeneous catalysts currently make it possible to obtain the desired coupling product under mild conditions and with a high yield. However, the catalytic systems that are used are relatively expensive, and the palladium catalyst cannot be recovered at the end of the reaction. To remedy these drawbacks, several heterogeneous catalysts that can be reused where the palladium is immobilized on inorganic or organic substrates have been developed (Choudary, B. M.; Madhi, S.; Chowdari, N. S.; Kantam, M. L.; Sreedhar, B. J. Am. Chem. Soc. 2002, 124, 14127. Mori, K.; Yamaguchi, T.; Hara, T.; Mizukagi, T.; Ebitani, K.; Kaneda, K. J. Am. Chem. Soc. 2002, 124, 11572. Bulut, H.; Artok, L.; Yilmaz, S. Tetrahedron Lett. 2003, 44, 289. Shimizu, K.-I.; Kanno, T.; Kodama, T.; Hagiwara, H.; Kitayama, Y. Tetrahedron Lett. 2002, 43, 5653. Baleizao, C.; Corma, A.; Garcia, H.; Leyva, A. Chem. Commun. 2003, 606. Zhang, T. Y.; Allen, M. J. Tetrahedron Lett. 1999, 40, 5813. Fenger, I.; Le Drian, C. Tetrahedron Lett. 1998, 39, 4287—this catalyst that has a palladium substrate is marketed by Fluka (catalyst No. 10987). Inada, K.; Miyaura, N. Tetrahedron Lett. 2000, 56, 8661. Parrish, C. A. Buchwald, S. L. J. Org. Chem. 2001, 66, 3820. Yamada, Y. M.; Takeda, K.; Takahashi, H.; Ikegami, S. J. Org. Chem. 2003, 68, 7733. Kang, T.; Feng, Q.; Luo, M. Synlett 2005, 15, 2305. Bedford, R. B.; Coles, S. J.; Hursthouse, M. B.; Scordia, V. J. M. J. Chem. Soc. Dalton Trans. 2005, 991. Lin, C.-A.; Luo, F.-T. Tetrahedron Lett. 2003, 44, 7565. Kim, J.-H.; Kim, J.-W.; Shokouhimehr, M.; Lee, Y.-S. J. Org. Chem. 2005, 70, 6714. Glegola, K.; Framery, E.; Pietrusiewicz, K. M.; Sinou, D. Adv. Synth. Catal. 2006, 348, 1728.).

The polymers constitute organic substrates of choice for synthesizing heterogeneous catalysts. Thus, in 2000, the use of PdCl₂ grafted on a diphenylphosphino ligand that has a polystyrene substrate for carrying out Suzuki couplings between an aryl chloride and an arylboronic acid was described (Inada, K.; Miyaura, N. Tetrahedron Lett. 2000, 56, 8661). This coupling requires, however, the use of large quantities of palladium (up to 30 mequivalents) and is primarily limited to the use of activated aryl chlorides (electro deficient) or chloropyridines.

In 2001, a five-stage synthesis of a dialkylphosphino ligand that has a polystyrene resin substrate was described. In the presence of palladium and under anhydrous reaction conditions, the latter makes it possible to carry out Suzuki couplings that use aryl bromides or aryl chlorides (Parrish, C. A.; Buchwald, S. L. J. Org. Chem. 2001, 66, 3820). In the case of reactions that invoke aryl chlorides, it is necessary to note a quite weak reactivity of the heterogeneous catalyst since it is necessary to use 10 mequivalents of palladium and up to 3 equivalents of boronic acid to obtain a quantitative yield. Finally, the possibility of recycling the heterogeneous catalyst had been studied only in the case of Suzuki couplings that invoke aryl bromides.

More recently, various palladacycles that have a polystyrene substrate that make it possible to couple aryl chlorides with boronic acids have been developed (Bedford, R. B.; Coles, S. J.; Hursthouse, M. B.; Scordia, V. J. M. J. Chem. Soc. Dalton Trans. 2005, 991). The major drawback of these heterogeneous catalysts is the impossibility of reusing them after reaction.

In 2006, a four-stage synthesis of a catalyst that comprises an aryldicyclohexyl-phosphine ligand that has a polymer substrate that makes it possible to carry out Suzuki couplings between activated (electrodeficient) aryl chlorides and phenylboronic acid or 4-methylphenylbornoic acid was developed (Glegola, K.; Framery, E.; Pietrusiewicz, K. M.; Sinou, D. Adv. Synth. Catal. 2006, 348, 1728). The use of a single deactivated aryl chloride was added thereto, and it leads to low and even zero yields.

There is therefore a real need for development of heterogeneous catalysts that can be reused with palladium and that are easy to access and that make it possible to carry out effective Suzuki couplings in particular between aryl chlorides and boronic acids.

This invention has as its object to remedy at least some of the above-mentioned drawbacks.

For this purpose, it has as its object new reusable palladium catalysts that have polymer substrates. The latter are quickly and easily synthesized on a large scale starting from commercial reagents and are effective for the Suzuki couplings, in particular those that involve aryl chlorides and preferably haloaryls and/or arylboronic acids that are activated as well as deactivated, in particular under non-anhydrous conditions.

This invention therefore has as its object a process for synthesis of a heterogeneous palladium catalyst that can catalyze a C—C coupling reaction, comprising the stages that essentially consist in making available a solid substrate on which there are fixed, in a covalent manner, groups of formula PR₁R₂, in which R₁ represents an optionally substituted alkyl group or an optionally substituted cycloalkyl group, and R₂ represents an optionally substituted aryl group or an optionally substituted heteroaryl group, and in incorporating a catalytically effective quantity of palladium in said substrate that is thus substituted.

The incorporation of said catalytically effective quantity of palladium in the above-mentioned substrate that is thus substituted is advantageously done at the above-mentioned groups of formula —PR₁R₂.

It also has as its object a heterogeneous catalyst that is obtained by the implementation of the process according to the invention, i.e., a heterogeneous palladium catalyst that is able to catalyze a C—C coupling reaction between two carbons sp² that comprise a solid substrate, preferably in the form of an organic polymer or copolymer, provided with at least one group —PR₁R₂, where R₁ and R₂ are as defined in this description, and provided with a catalytically adequate quantity of palladium, advantageously fixed at said group or groups —PR₁R₂.

Finally, it also has as its object the use of a catalyst according to the invention for catalyzing a Suzuki coupling reaction and particularly between an aryl halide or a heteroaryl halide, and an arylboronic acid or a heteroarylboronic acid, whereby said aryl halide or heteroaryl halide and/or the arylboronic acid or heteroarylboronic acid can carry one or more electron-donor or electron-attractor substituents, and whereby said halide is preferably a chloride.

The invention will be better understood, using the description below, which relates to preferred embodiments, provided by way of nonlimiting examples.

This invention therefore has as its object a process for synthesis of a heterogeneous palladium catalyst that can catalyze a C—C coupling reaction, comprising the stages that essentially consist in making available a solid substrate to which are fixed in a covalent manner groups of formula —PR₁R₂, in which R₁ represents an optionally substituted alkyl group, or an optionally substituted cycloalkyl group, and R₂ represents an optionally substituted aryl group or an optionally substituted heteroaryl group, and in incorporating a catalytically effective quantity of palladium in said thus substituted substrate.

The incorporation of said catalytically effective quantity of palladium in the above-mentioned substrate that is thus substituted advantageously is done at the above-mentioned groups of the formula —PR₁R₂, in particular by the formation of a complex —PR₁R₂Pd.

The term alkyl refers to a hydrocarbon chain, linear or branched, containing 1 to 20 carbon atoms, preferably 1 to 12 carbon atoms, more preferably 1 to 8 carbon atoms, and most preferably a tert-butyl group.

The term cycloalkyl refers to a monocyclic, bicyclic or tricyclic hydrocarbon compound that comprises 3 to 11 carbon atoms, and is optionally unsaturated by 1 or 2 unsaturations.

For R₂, the term aryl refers to a group that comprises at least one aromatic core and that comprises 6 to 20 carbon atoms, preferably 6 to 10 carbon atoms, and more preferably a group that is selected from the group that is formed by the groups phenyl, naphthyl, 2-methylphenyl, 3-methylphenyl, or 4-methylphenyl.

For R₂, the term heteroaryl refers to a monocyclic or bicyclic group in which at least one of the cycles is aromatic, whereby said group comprises 5 to 11 links and 1 to 4 heteroatoms that are selected from among nitrogen, oxygen and sulfur.

The expression “optionally substituted” that is assigned to the terms alkyl, cycloalkyl, aryl or heteroaryl means that these groups can be substituted by one to four substituents that are identical or different that are selected from among the following groups: alkyl, alkoxy, alkylthio, aryl, monoalkylamino or dialkylamino, where
alkyl refers in turn to a hydrocarbon chain, linear or branched, containing 1 to 20 carbon atoms, preferably 1 to 12 carbon atoms, and more preferably 1 to 8 carbon atoms,
alkoxy, alkylthio, monoalkylamino or dialkylamino refers to an alkyl-oxy, alkyl-thio, alkyl-amino or dialkyl-amino group whose alkyl chain or chains, linear or branched, each contain(s) 1 to 8 carbon atoms, and
aryl refers to a group that comprises at least one aromatic core and that comprises 6 to 20 carbon atoms, preferably 6 to 10 carbon atoms.

Advantageously, the solid substrate is an organic polymer or an organic copolymer, and preferably the organic substrate comprises or is a copolymer of styrene and divinylbenzene.

According to one variant, the organic substrate comprises or is a polystyrene block copolymer and an ethylene poly(oxide) block copolymer.

According to a first aspect, the process according to the invention is characterized in that R₁ represents an optionally substituted alkyl group, or an optionally substituted cycloalkyl group, and R₂ represents an optionally substituted aryl group, or an optionally substituted heteroaryl group, and in incorporating a catalytically effective quantity of palladium in said thus substituted substrate. The incorporation of said palladium can be done at the above-mentioned groups of formula —PR₁R₂.

Advantageously, R₁ represents a C₁ to C₂₀ alkyl group, preferably a C₁ to C₁₂ alkyl group, more preferably a C₁ to C₈ alkyl group, and, most preferably, a tert-butyl group.

Furthermore, the process according to the invention is characterized in that R₂ is a C₆ to C₂₀ aryl group, preferably a C₆ to C₁₂ aryl group, more preferably a C₆ to C₁₀ aryl group, and, most preferably, a group that is selected from the group that is formed by the phenyl, naphthyl, 2-methylphenyl, 3-methylphenyl or 4-methylphenyl groups.

The process according to the invention is also characterized in that the palladium is incorporated by treating the solid substrate that has said groups with formula —PR₁R₂ with a solution of at least one salt or at least one palladium complex, preferably a solution of Pd(PPh₃)₄, so as to obtain a palladium content in the substrate catalyst that is less than or equal to 5% by mass of said substrate catalyst.

According to a particular embodiment that is described in more detail below, the process according to the invention is characterized in that prior to the palladium incorporation treatment, a solid substrate that consists essentially of a partially halogenated synthetic resin is made available, in that at least a portion of the halogen atoms of said substrate is substituted by a compound of general formula R₁R₂PLi, and then in that the palladium is incorporated in said substituted substrate that is thus obtained, preferably by treating it with a solution that contains said palladium.

Advantageously, the synthetic resin is chlorinated and/or brominated.

The invention also has as its object a heterogeneous palladium catalyst that is obtained by the implementation of the process according to the invention, namely a heterogeneous palladium catalyst that is able to catalyze a C—C coupling reaction between two carbons sp² that comprise a solid substrate, preferably in the form of an organic polymer or copolymer, provided with at least one group —PR₁R₂, where R₁ and R₂ are as defined in this description, and provided with a catalytically adequate quantity of palladium that is fixed at said group or groups —PR₁R₂.

Preferably, the catalyst that is obtained by the implementation of the process according to the invention is characterized in that:

R₁ is a tert-butyl group, and R₂ is a phenyl group,

R₁ is a tert-butyl group, and R₂ is a 2-methylphenyl group,

R₁ is a tert-butyl group, and R₂ is a 3-methylphenyl group,

R₁ is a tert-butyl group, and R₂ is a 4-methylphenyl group,

R₁ is a tert-butyl group, and R₂ is a naphthyl group, or

R₁ is a tert-butyl group, and R₂ is a tert-butyl group.

Advantageously, the substrate is a polystyrene resin, preferably a resin that is known under the name “Merrifield polystyrene resin,” or a polystyrene and ethylene poly(oxide) resin, preferably a resin that is known under the name “Tentagel resin.”

This invention also has as its object the use of a catalyst according to the invention for catalyzing a Suzuki coupling reaction between an aryl halide or a heteroaryl halide and an arylboronic acid or heteroarylboronic acid, whereby said aryl halide or heteroaryl halide and/or the arylboronic or heteroarylboronic acid can carry one or more electron-donor or electron-attractor substituents, and whereby said halide is preferably a chloride.

“Arylboronic” or “heteroarylboronic” is defined as an aryl or heteroaryl group as defined above for R₂ and whereby each has a group —B(OH)₂.

Preferably, the coupling reaction is carried out in a solvent that is based on toluene and water, under a temperature of between 65° C. and 110° C. and in the presence of at least one alkaline fluoride, preferably in the presence of cesium fluoride.

According to another aspect, the coupling reaction is carried out with the addition of at least one carbonated base, preferably cesium carbonate and/or sodium carbonate.

Preferably, the use according to the invention is characterized in that the aryl chloride is 4-chloroacetophenone, optionally substituted by one or more electron-donor or electron-attractor groups, by the fact that the aryl chloride is 2-chloropyridine, optionally substituted by one or more electron-donor or electron-attractor groups, or by the fact that the aryl chloride is chlorobenzene, optionally substituted by one or more electron-donor or electron-attractor groups.

Likewise, the use is characterized in that the arylboronic acid is the phenylboronic acid that is optionally substituted by one or more electron-donor or electron-attractor groups or in that the heteroarylboronic acid is the 3-thiopheneboronic acid that is optionally substituted by one or more electron-donor or electron-attractor groups.

Advantageously, in the coupling reaction, a quantity of palladium substrate that is contained in the catalyst of between 0.01 mequivalent and 5 mequivalents is used.

Within the scope of this invention, in particular catalysts with palladium substrate on a gel-type resin were synthesized by using a Merrifield polystyrene resin (PS-CH₂Cl resin) or a brominated Tentagel resin (PS-PEG-Br resin), both of the two available commercially. The latter allow good accessibility to the active sites for the pallado-catalyzed reactions that take place in hot aromatic solvents. Within the scope of the invention, the grafting of alkylarylphosphino ligands has been studied.

In a practical way, the synthesis of the substrate catalysts can be carried out, for example, in two stages: grafting of a phosphate ligand by substitution of the halogen atom of the Merrifield resin or the Tentagel resin by an alkylaryl-phosphino-lithium R₁R₂PLi (where R₁ and R₂ are as defined previously), followed by the introduction of palladium using a soluble palladium complex (diagram 1).

Additional studies, produced by the applicant on catalysts that have a palladium substrate on diarylphosphinopolystyrenes, led to poor results in the Suzuki coupling between aryl chlorides and arylboronic acids.

The R₁R₂PLi compounds that are referred to above and that are used within the scope of a particularly preferred process can, for example, be obtained from lithium and chlorophosphines R₁R₂PCl (where R₁ and R₂ are as defined previously). The latter can be generated by reaction of an alkyl- or an aryl-lithine with a dichloroaryl- or dichloroalkyl-phosphine. By way of example, reaction conditions have been developed by using—as model substrates—tert-butyllithium and dichlorophenylphosphine, both common and commercially available products. One particularly effective synthesis process, provided by way of nonlimiting example, consists in adding at −40° C. one equivalent of dichlorophenylphosphine to a solution of tert-butyllithium in cyclohexane. Since the chlorophosphines are often sensitive to air, their purification is often difficult and tedious. A procedure was then developed that makes possible the direct implementation of the next stage. Thus, after reaction between the tert-butyllithium and the dichlorophenylphosphine, the reaction medium is centrifuged to eliminate the lithium salts, the supernatant is removed, and the solvents are eliminated by distillation.

The thus obtained crude chlorophosphine has a purity of greater than 90% (determined by ³¹P and ¹H NMR) and can be engaged directly in the chlorine-lithium exchange stage to generate the compound R₁R₂PLi. The latter then reacts with the Merrifield resin at 25° C. for 72 hours.

The palladium is finally introduced into the polymer by reaction with Pd(PPh₃)₄ at 100° C. in toluene to lead to the C1 substrate catalyst (diagram 1).

Elementary analyses that are carried out on the catalyst C1 have shown that more than 95% of the quantity of palladium that is introduced is fixed to the polymer substrate. This reaction sequence has easily been transposed to the synthesis of 10 g of the catalyst C1. It is also suitable to note that the catalyst C1 that is thus obtained is perfectly stable against air and humidity and does not require any special precautions for use and storage.

The synthesis methodology described above was then successfully transposed in the preparation of substrate catalysts C2-C5 (cf. diagrams 1 and 2). This catalyst C5 comprises a phosphorus atom that has identical groups R₁ and R₂ and is not part of this invention.

Furthermore, the catalyst C6, analogously to C1, was prepared by replacing the so-called “Merrifield” polymer by a brominated “Tentagel” polymer (cf. diagram 2).

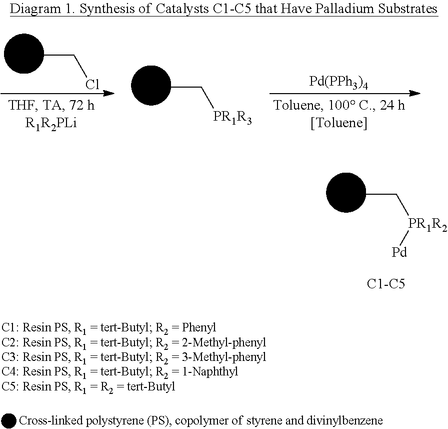

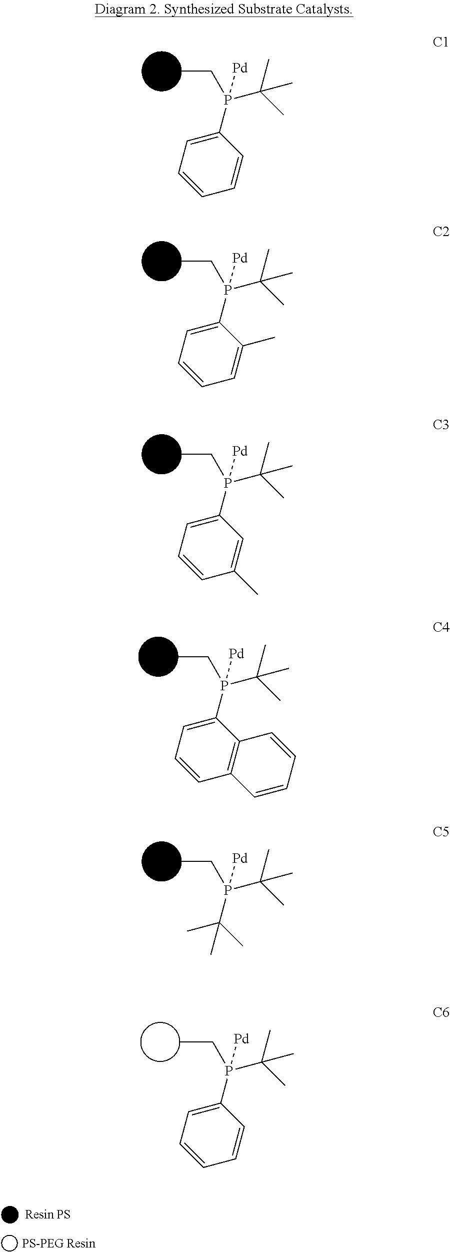

The reaction conditions have been developed by using—as examples of substrates—4-chloroacetophenone, phenylboronic acid (in the solid state, the phenylboronic acid is in the form of trimeric boroxine, which is transformed in aqueous medium into acid), and the catalyst C1.

The Suzuki couplings have been carried out in a mixture of toluene/EtOH/H₂O 5:1:1 (by volume) by using sodium carbonate as a base. The yields of the reaction crude were estimated by ¹H NMR (cf. Table 1).

TABLE 1
Pallado-Catalyzed Coupling between 4-Chloroacetophenone and Phenylboronic
Acid

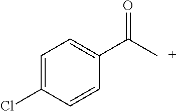

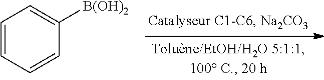

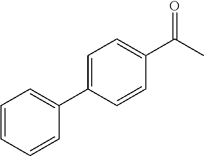

| | | | Quantity of | | |
|---|---|---|---|---|---|
| | | Content by Mass of | Catalyst | | Estimated |
| | | Pd of the Catalyst | (mequivalents of | | Yield |
| Entry | Catalyst | (%)⁽¹⁾ | Pd)⁽²⁾ | Temperature (° C.) | (%)⁽³⁾ |
| 1 | C1 | 0.3 | 2.0 | 100 | 100 |
| 2 | C1 | 0.3 | 1.0 | 100 | 73 |
| 3 | C1 | 0.1 | 0.5 | 100 | 100 |
| 4 | C1 | 0.1 | 0.1 | 100 | <20 |
| 5 | C2 | 0.3 | 2.0 | 100 | 14 |
| 6 | C3 | 0.3 | 2.0 | 100 | 95 |
| 7 | C4 | 0.3 | 2.0 | 100 | 18 |
| 8 | C5 | 0.3 | 2.0 | 100 | 84 |
| 9 | C6 | 0.1 | 0.5 | 100 | 100 |
| 10 | C6 | 0.1 | 0.1 | 100 | 98 |
| 11 | C6 | 0.1 | 0.05 | 100 | 87 |
| 12 | C6 | 0.1 | 0.5 | 65 | 90 |

[Key to Table 2:]
Catalyseur = Catalyst
Toluène = Toluene
⁽¹⁾The palladium content of the catalyst was determined by elementary analysis.
⁽²⁾Use of 1.0 equivalent of 4-chloroacetophenone, 1.1 equivalents of phenylboronic acid, 1.2 equivalents of Na₂CO₃, and the indicated quantity of palladium.
⁽³⁾The yields of the reaction crude were calculated by 1H NMR.

The catalyst C1 that has a resin substrate PS and that comprises 0.3% by mass of palladium makes it possible to obtain a total reaction between the 4-chloroacetophenone and phenylboronic acid in the presence of 2.0 mequivalents of palladium substrate (entry 1). The use of 1.0 mequivalent of palladium leads to a drop in yield to 73% (input 2). A better reactivity of the catalyst C1 is observed when the quantity of palladium that is grafted on the polymeric substrate is only 0.1%. In this case, the yield is also quantitative in the presence of 0.5 mequivalent of palladium substrate (entry 3). A reduction of the quantity of palladium to 0.1 mequivalent produces a reduction in yield, however (entry 4). The catalysts C2-C5 that are more encumbered than C1 are less effective in the presence of 2.0 mequivalents of palladium substrate (entries 1 and 5-8). The nature of the polymeric substrate was then modified by replacing the substrate PS by a substrate PS-PEG. It is noted that the corresponding catalyst C6 is as effective as C1 in the presence of 0.5 mequivalent of palladium (entries 3 and 9). The catalyst C6, however, offers a better reactivity than C1 when the quantity of palladium that is introduced is only 0.1 mequivalent; a yield of 98% is thus obtained (entries 4 and 10). By using 0.05 mequivalent of palladium (C6), the yield is still high and reaches 87% (entry 11). Finally, a reduction of the temperature to 65° C. leads to a slight reduction in yield (entry 12).

### Recycling of Palladium Catalysts with Polymer Substrates

The possibility of recycling and then reusing the catalyst has also been studied within the framework of this invention. Thus, after the Suzuki coupling reaction, the catalyst is filtered and then washed and finally dried under vacuum. It is then reused in a new Suzuki coupling between the 4-chloroacetophenone and the phenylboronic acid. Several reaction conditions (A-E) have been developed and then tested (cf. Table 3).

TABLE 2
Tests for Recycling C1 and C6 Catalysts.

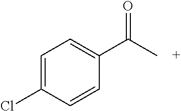

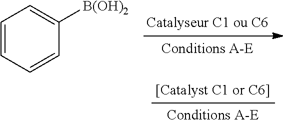

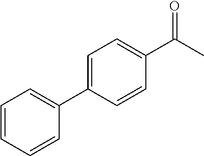

| Use⁽¹⁾ | 1^{st} | 2^{nd} | 3^{rd} | 4^{th} | 5^{th} | 6^{th} | 7^{th} |
|---|---|---|---|---|---|---|---|
| Conditions A⁽²⁾ | 100 | 32 | <10 | — | — | — | — |
| Conditions B⁽²⁾ | 88 | 75 | — | — | — | — | — |
| Conditions C⁽²⁾ | 100 | 87 | — | — | — | — | — |
| Conditions D⁽²⁾ | 94 | 95 | 100 | 98 | 100 | 98 | 98 |
| Conditions E⁽²⁾ | 100 | 100 | 97 | 84 | 65 | — | — |

Conditions A: PhB(OH)₂ (1.1 equivalents), Na₂CO₃ (1.2 equivalents), 0.5 mequivalent of Pd(C1), toluene/EtOH/H₂O 5:1:1, 100° C., 20 hours
Conditions B: PhB(OH)₂ (1.4 equivalents), Cs₂CO₃ (1.55 equivalents), 2.0 mequivalents of Pd(C1), toluene (+10 μl of H₂O), 100° C., 20 hours
Conditions C: PhB(OH)₂ (1.4 equivalents), Cs₂CO₃ (1.55 equivalents), 3.0 mequivalents of Pd(C1), toluene (+10 μl of H₂O), 65° C., 20 hours
Conditions D: PhB(OH)₂ (1.4 equivalents), CsF (1.55 equivalents), 4.0 mequivalents of Pd(C1), Toluene (+10 μl of H₂O), 100° C., 20 hours
Conditions E: PhB(OH)₂ (1.1 equivalents), Na₂CO₃ (1.2 equivalents), 1.0 mequivalent of Pd (C6), toluene/EtOH/H₂O 5:1:1, 100° C., 20 hours
⁽¹⁾Catalyst C1 or C6 at 0.1% by mass of palladium.
⁽²⁾The yields of the reaction crude were calculated by ¹H NMR.

By using the reaction conditions that were previously developed (conditions A), a reduction in yield is observed during the second use of the substrate catalyst C1. Stability tests of C1 were then carried out to explain the drop in yield that is observed. For this purpose, C1 was first heated to 100° C. in a mixture of toluene/EtOH/H₂O for 20 hours. Then, the 4-chloroacetophenone, phenylboronic acid and Na₂CO₃ were successively added, and the reaction medium was heated at 100° C. for 20 hours. The yield that is observed is then 30%. This result seems to show that the catalyst C1 degrades in a hot protic solvent. In this case, it is suitable to use different reaction conditions that make it possible to use C1 in a non-protic solvent.

For this purpose, it was shown that the Suzuki coupling reaction between the 4-chloroacetophenone and the phenylboronic acid can also be carried out in toluene at 100° C. or at 65° C. by using Cs₂CO₃ as a base (table 2, conditions B and C) in the presence of traces of water (10 μl). The yield of the coupling after the second use of C1 is thus considerably improved.

Better recycling results can be obtained by using CsF as a base (Table 2, conditions D): the catalyst C1 can thus be employed more than seven times with a yield that is higher than 94% with each use.

Furthermore, it was shown that the losses of palladium in the presence of CsF (conditions D) are only on the order of 0.1% by mass of the quantity of palladium that is introduced. Under these conditions, the use of smaller quantities of palladium seems to provoke a decrease in yield.

The recycling of the catalyst C6, which offers a better affinity for the protic solvents, was also evaluated (Table 2, conditions E), whereby the yield of the Suzuki coupling after the fourth use is still 87%.

### Extension to the Use of Other Arylboronic Acids and Aryl Chlorides

By way of nonlimiting example, the use of the substrate catalyst C1 that comprises 0.1% by mass of palladium has been extended to Suzuki coupling between various aryl chlorides and various boronic acids (cf. Table 3).

TABLE 3
Pallado-Catalyzed Couplings between Various Aryl Chlorides and Arylboronic
Acids.

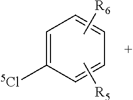

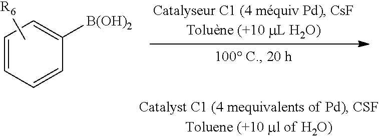

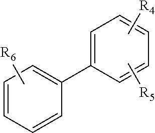

| | | | | Isolated Yield |
|---|---|---|---|---|
| Entry⁽¹⁾ | R₄ | R₅ | R₆ | (%)⁽²⁾ |
| 1 | 4-Ac | H | H | 90 |
| 2⁽³⁾ | 4-Ac | H | 3-NO₂ | 78 |
| 3 | 4-Ac | H | 4-Me | 90 |
| 4 | 4-Ac | H | 2-Me | 86 |
| 5 | 4-Ac | H | 4-OMe | 93 |
| 6⁽³⁾ | 4-Ac | H | 3-NH₂ | 69 |
| 7 | 3-Ac | H | H | 78 |
| 8 | 2-Ac | H | H | 90 |
| 9 | 4-NO₂ | H | H | 86 |
| 10 | H | H | H | 98 |
| 11 | 4-OMe | H | H | 72 |
| 12 | 4-Me | H | H | 86 |
| 13 | 3-Me | H | H | 79 |
| 14 | 2-Me | H | H | 82 |
| 15 | 2-Me | 6-Me | H | 88 |

⁽¹⁾Use of 1.0 equivalent of aryl chloride, 1.4 equivalents of boronic acid, 1.55 equivalents of CsF, and 4 mequivalents of Pd.
⁽²⁾Isolated yield, calculated after purification of the reaction crude on a silica gel column.
⁽³⁾Addition of 0.5 ml of EtOH in the reaction medium.

The substrate catalyst C1 (0.1% palladium) can be used successfully for the Suzuki coupling of various arylboronic acids with 4-chloroacetophenone (entries 1-6). Furthermore, this coupling can be extended to the use of various aryl chlorides that carry other electron-attractor substituents (entries 7-9) or electron-donor substituents (entries 11-15). In a noteworthy manner, couplings that cause encumbered substrates to occur are also possible (entries 4, 14, and primarily 15).

In most cases, a simple filtration on silica gel makes it possible to purify the coupling product. By using the previously developed reaction conditions, the Suzuki coupling also has been successfully extended with the use of 2-chloropyridine and 3-thiopheneboronic acid with yields of 88% and 64% respectively (diagram 3).

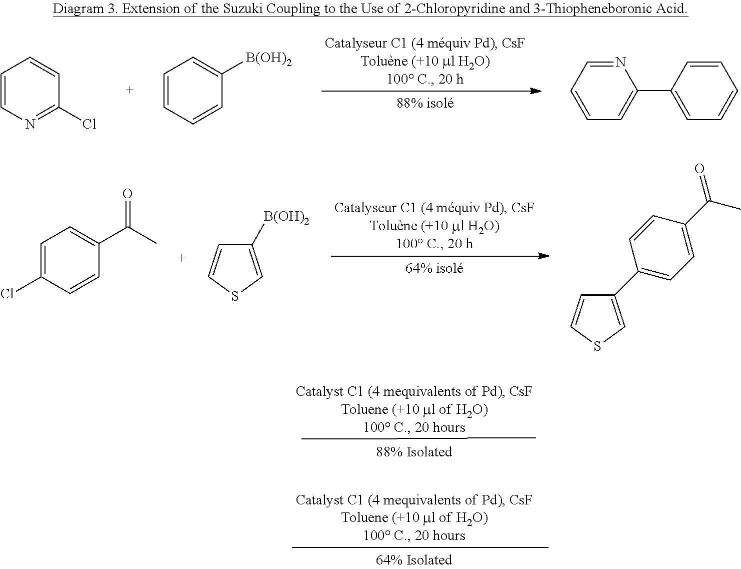

The use of aryl chlorides instead of aryl bromides or aryl iodides in the Suzuki couplings offers a certain advantage in organic synthesis so as to reduce production costs on an industrial scale.

The heterogeneous palladium catalysts of this invention can, contrary to some of the current catalysts, easily be prepared on a large scale from commercial products. They make it possible in particular to produce effective Suzuki couplings between aryl chlorides and boronic acids. The quantities of palladium that are involved are relatively small, and the coupling yields are comparable to those that are described by Buchwald (Walker, S. D. et al. Angew. Chem. Int. Ed. 2004, 43, 1871).

Finally, the developed heterogeneous catalysts can easily be recycled, and then reused more than seven times without lowering the yield.

### Preparation of the Catalyst C1

a) Synthesis of tert-Butylchlorophenylphosphine.

Dichlorophenylphosphine (26.0 mmol, 3.53 ml, 1.0 equivalent) is added in several portions at −40° C. to a 1.5 M tert-butyllithium solution in pentane (31.2 mmol, 20.8 ml, 1.2 equivalents) mixed with anhydrous cyclohexane (0 ml) under argon atmosphere. The reaction medium is stirred at −40° C. for one hour, and then at ambient temperature for 20 hours. The reaction medium is then centrifuged (3000 rpm for 3 minutes) under argon atmosphere. The supernatant is transferred into a heated flask under an argon atmosphere. The solvents are distilled under argon, and the chlorophosphine is dried under vacuum (0.1 mbar) for 20 hours. 31P and 1H NMR analyses carried out on crude chlorophosphine showed that the latter is obtained with a purity of more than 90%. Said chlorophosphine is directly engaged in the following reaction of chlorine-lithium exchange.

b) Synthesis of (tert-Butyl)phenylphosphinopolystyrene

The crude chlorophosphine (26.0 mmol, 10.1 equivalents) that is obtained previously is diluted in anhydrous THF (60 ml), and then it is added to lithium chips (78.0 mmol, 540 mg, 30.2 equivalents) under argon atmosphere. The reaction medium is stirred at ambient temperature for 20 hours. The red anion solution in the THF is then transferred to a Merrifield resin suspension (feedstock: 0.86 mmol.g⁻¹ of chlorine, 3 g, 1 equivalent) in anhydrous THF (60 ml) under argon atmosphere. The reaction medium is stirred at ambient temperature for 72 hours. It is then neutralized by adding an acetone/H₂O mixture that is 2:1 by volume (30 ml). The resin is filtered under vacuum and washed successively with water, acetone, chloroform, toluene, and diethyl ether. The thus obtained resin is then reflux-heated in an EtOH/toluene mixture that is 3:1 by volume (50 ml) for 20 hours. After cooling to ambient temperature, the resin is filtered under vacuum, washed with toluene and then with ether, and finally dried under vacuum (0.1 mbar) for 20 hours.

c) Synthesis of the Substrate Catalyst C1 (at 0.1% by Mass of Palladium)

The previously obtained resin (2.60 g) is suspended in anhydrous toluene (120 ml) under argon atmosphere. Pd(PPh₃)₄ (28.1 mg) is added at one time. The reaction medium is degassed, placed under argon atmosphere, and finally reflux-heated for 20 hours. After cooling to ambient temperature, the catalyst C1 is washed with toluene (3 times), and then with diethyl ether (3 times). It is finally dried under vacuum (0.1 mbar) for 20 hours. The substrate catalyst C1 (pale yellow resin) that is perfectly stable against air and humidity is thus obtained.

d) Suzuki Coupling between 4-Chloroacetophenone and Phenylboronic Acid

The previously prepared substrate catalyst C1 (277 mg, 4 mequivalents of palladium, resin at 0.1% by mass of palladium) is added to a solution of 4-chloroacetophenone (0.65 mmol, 84 μl, 1.0 equivalent), phenylboronic acid (0.91 mmol, 111 mg, 1.4 equivalents), and CsF (1.01 mmol, 153 mg, 1.55 equivalents) in a toluene mixture (3.5 ml)/H₂O (10 μl). The reaction medium is degassed under argon and then heated to 100° C. for 20 hours. After cooling to ambient temperature, the catalyst C1 is filtered under vacuum and rinsed 3 times with ethyl acetate (3×20 ml). The organic phase is washed with water, dried on MgSO₄, and then reconcentrated under vacuum. The crude reaction mixture is finally filtered on silica gel to obtain 4-phenylacetophenone (0.62 mmol, 122 mg, white solid) with an isolated yield of 96%.

e) Losses of Palladium

Losses of palladium are determined in the following way: the crude reaction mixture is evaporated under reduced pressure, the residue is attacked by concentrated H₂SO₄, and fuming HNO₃ under reflux, and the palladium is metered into the aqueous solution that is finally obtained.

Of course, the invention is not limited to the embodiments that are described. Modifications are possible, in particular from the standpoint of the composition of various elements or by substitution of technical equivalents, without thereby exceeding the scope of protection of the invention.

1) Process for synthesis of a heterogeneous palladium catalyst that can catalyze a C—C coupling reaction, comprising the stages that essentially consist in making available a solid substrate on which there are fixed, in a covalent manner, groups of formula PR₁R₂, in which R₁ represents an optionally substituted alkyl group or an optionally substituted cycloalkyl group, and R₂ represents an optionally substituted aryl group or an optionally substituted heteroaryl group, and in incorporating a catalytically effective quantity of palladium in said substrate that is thus substituted.

2) Process according to claim 1, in which the solid substrate is an organic polymer or an organic copolymer.

3) Process according to claim 1, characterized in that the organic substrate comprises or is a copolymer of styrene and divinylbenzene.

4) Process according to claim 1, wherein the organic substrate comprises or is a copolymer with blocks of polystyrene and ethylene poly(oxide).

5) Process according to claim 1, wherein R₁ represents a C₁ to C₂₀ alkyl group, preferably a C₁ to C₁₂ alkyl group, more preferably a C₁ to C₈ alkyl group, and, most preferably, a tert-butyl group.

6) Process according to claim 1, wherein R₂ is a C₆ to C₂₀ aryl group, preferably a C₆ to C₁₂ aryl group, more preferably a C₆ to C₁₀ aryl group, and, most preferably, a group that is selected from the group that is formed by the phenyl, naphthyl, 2-methylphenyl, 3-methylpheyl or 4-methylphenyl groups.

7) Process according to claim 1, wherein the palladium is incorporated by treating the solid substrate that has said groups of formula —PR₁R₂ with a solution of at least one salt or at least one palladium complex, preferably a solution of Pd(PPh₃)₄, so as to obtain a palladium content in the substrate catalyst that is less than or equal to 5% by mass of said substrate catalyst.

8) Process according to claim 7, wherein prior to the palladium incorporation treatment, a solid substrate that consists essentially of a partially halogenated synthetic resin is made available, wherein at least a portion of the halogen atoms of said substrate is substituted by a compound of general formula R₁R₂PLi, and then wherein the palladium is incorporated in said substituted substrate that is thus obtained, preferably by treating it with a solution that contains said palladium.

9) Process according to claim 8, wherein the synthetic resin is chlorinated and/or brominated.

10) Heterogeneous palladium catalyst that is obtained by the implementation of the process according to claim 1.

11) Catalyst that is obtained by the implementation of the process according to claim 1, wherein R₁ is a tert-butyl group and R₂ is a phenyl group.

12) Catalyst that is obtained by the implementation of the process according to claim 1, wherein R₁ is a tert-butyl group, and R₂ is a 2-methylphenyl group.

13) Catalyst that is obtained by the implementation of the process according to claim 1, wherein R₁ is a tert-butyl group and R₂ is a 3-methylphenyl group.

14) Catalyst that is obtained by the implementation of the process according to claim 1, wherein R₁ is a tert-butyl group and R₂ is a 4-methylphenyl group.

15) Catalyst that is obtained by the implementation of the process according to claim 1, wherein R₁ is a tert-butyl group, and R₂ is a naphthyl group.

16) Catalyst that is obtained by the implementation of the process according to claim 1, wherein R₁ is a tert-butyl group, and R₂ is a tert-butyl group.

17) Catalyst according to claim 10, wherein the substrate is a polystyrene resin, preferably a resin that is known under the name “Merrifield polystyrene resin.”

18) Catalyst according to claim 10, wherein the substrate is a polystyrene and ethylene poly(oxide) resin, preferably a resin that is known under the name “Tentagel resin.”

19) Method for catalyzing a Suzuki coupling reaction such as between an aryl halide or a heteroaryl halide and an arylboronic acid or heteroarylboronic acid, whereby said aryl halide or heteroaryl halide and/or arylboronic acid or heteroarylboronic acid can carry one or more electron-donor or electron-attractor substituents and whereby said halide is preferably a chloride, which comprises using a catalyst according to claim 10.

20) Method according to claim 19, wherein the reaction is carried out in a solvent that is based on toluene and water, under a temperature of between 65° C. and 110° C. and in the presence of at least one alkaline fluoride, preferably in the presence of cesium fluoride.

21) Method according to claim 19, wherein the reaction is carried out with the addition of at least one carbonated base, preferably cesium and/or sodium carbonate.

22) Method according to claim 19, wherein the aryl chloride is 4-optionally substituted by one or more electron-donor or electron-attractor groups.

23) Method according to claim 19, wherein the aryl chloride is 2-optionally substituted by one or more electron-donor or electron-attractor groups.

24) Method according to claim 19, wherein the aryl chloride is chlorobenzene, optionally substituted by one or more electron-donor or electron-attractor groups.

25) Method according to claim 19, wherein the arylboronic acid is phenylboronic acid that is optionally substituted by one or more electron-donor or electron-attractor groups.

26) Method according to claim 19, wherein the heteroarylboronic acid is the 3-thiopheneboronic acid that is optionally substituted by one or more electron-donor or electron-attractor groups.

27) Method according to claim 19, wherein a quantity of palladium with a substrate that is contained in the catalyst of between 0.01 mequivalent and 5 mequivalents is used.
